(19) United States
(12) Patent Application Publication
(10) Pub. No.: US 2024/0293177 A1
(43) Pub. Date: Sep. 5, 2024

(54) SYSTEMS AND METHODS FOR AUTOMATIC DETECTION OF PHRENIC NERVE STIMULATION

(71) Applicant: St. Jude Medical, Cardiology Division, Inc., St. Paul, MN (US)

(72) Inventors: Travis Dahlen, Forest Lake, MN (US); Therese C. Gilbert, St. Paul, MN (US)

(21) Appl. No.: 18/591,636

(22) Filed: Feb. 29, 2024

Related U.S. Application Data
(60) Provisional application No. 63/449,373, filed on Mar. 2, 2023.

(51) Int. Cl. A61B 18/14; A61B 18/00

(52) U.S. Cl. CPC A61B 18/1492; A61B 2018/00351; A61B 2018/00577; A61B 2018/00613; A61B 2018/00642; A61B 2018/00672; A61B 2018/0072; A61B 2018/00773; A61B 2018/00904

(57) ABSTRACT

Systems and methods for automatic detection of phrenic nerve stimulation are disclosed. A plurality of pacing pulses are delivered between an associated pair of electrodes of a plurality of electrodes. For each of the plurality of pacing pulses, a corresponding diaphragm movement during delivery of the pacing pulse is measured. For each of the plurality of pacing pulses, it is determined, based on the corresponding measured diaphragm movement, whether the pacing pulse results in phrenic nerve capture. For each pacing pulse that results in phrenic nerve capture, the pair of electrodes associated with that pacing pulse is recorded.

### CROSS REFERENCE TO RELATED APPLICATIONS

This application claims priority to U.S. Provisional Patent Application No. 63/449,373 filed on Mar. 2, 2023, which is incorporated by reference herein in its entirety.

### FIELD OF THE DISCLOSURE

The present disclosure relates generally to tissue ablation systems and methods. In particular, the present disclosure relates to electroporation systems and methods with automatic phrenic nerve monitoring.

### BACKGROUND

It is generally known that ablation therapy may be used to treat various conditions afflicting the human anatomy. For example, ablation therapy may be used in the treatment of atrial arrhythmias. When tissue is ablated, or at least subjected to ablative energy generated by an ablation generator and delivered by an ablation catheter, lesions form in the tissue. Electrodes mounted on or in ablation catheters are used to cause tissue destruction in cardiac tissue to correct conditions such as atrial arrhythmia (including, but not limited to, ectopic atrial tachycardia, atrial fibrillation, and atrial flutter).

Arrhythmia (i.e., irregular heart rhythm) can create a variety of dangerous conditions including loss of synchronous atrioventricular contractions and stasis of blood flow which can lead to a variety of ailments and even death. It is believed that the primary cause of atrial arrhythmia is stray electrical signals within the left or right atrium of the heart. The ablation catheter imparts ablative energy (e.g., radiofrequency energy, cryoablation, lasers, chemicals, high-intensity focused ultrasound, etc.) to cardiac tissue to create a lesion in the cardiac tissue. This lesion disrupts undesirable electrical pathways and thereby limits or prevents stray electrical signals that lead to arrhythmias.

Electroporation is a non-thermal ablation technique that involves applying strong electric-fields that induce pore formation in the cellular membrane. The electric field may be induced by applying a relatively short duration pulse which may last, for instance, from a nanosecond to several milliseconds. Such a pulse may be repeated to form a pulse train. When such an electric field is applied to tissue in an in vivo setting, the cells in the tissue are subjected to trans-membrane potential, which opens the pores on the cell wall. Electroporation may be reversible (i.e., the temporally opened pores will reseal) or irreversible (i.e., the pores will remain open). For example, in the field of gene therapy, reversible electroporation (i.e., temporarily open pores) is used to transfect high molecular weight therapeutic vectors into the cells. In other therapeutic applications, a suitably configured pulse train alone may be used to cause cell destruction, for instance by causing irreversible electroporation.

For example, pulsed field ablation (PFA) may be used to perform instantaneous pulmonary vein isolation (PVI). PFA generally involves delivering high voltage pulses from electrodes disposed on a catheter. For example, voltage pulses may range from less than about 500 volts to about 3,000 volts or higher. These fields may be applied between pairs of electrodes (bipolar therapy) or between one or more electrodes and a return patch (monopolar therapy).

Although PFA is non-thermal and demonstrates a high degree of tissue selectivity, which may improve the safety of ablation procedures, there are still practical challenges that must be considered to maintain the improved safety profile. For example, the voltage threshold for cardiac cells is approximately 400 Volts/centimeter (V/cm). In some PFA systems, the therapy spans between electrodes and protrudes deep into the tissue, which may result in voltage gradients surpassing 400 V/cm at locations near the electrodes. If these electrodes are proximate to the phrenic nerve, which provides innervation for the diaphragm, there is a potential for acute phrenic stunning and/or long-term phrenic damage. Accordingly, to reduce the potential for phrenic impact, a physician may avoid ablating in areas near the phrenic nerve or use a lower dosage when ablating near the phrenic nerve.

Traditional methods for detecting phrenic nerve stimulation include placing electrodes in the heart, generating a pacing stimulus in the electrodes, and visually assessing diaphragm stimulation. If there is evidence of diaphragm capture, the physician may assume that the stimulated electrodes are on or near the phrenic nerve. The physician may then use this information to select a lower dosage waveform or to disable specific electrodes. Although straightforward, these traditional methods are time consuming and may be imprecise. As such, there is a need for more efficient and accurate phrenic nerve detection systems and methods.

### BRIEF SUMMARY OF THE DISCLOSURE

In one aspect, a method for phrenic nerve stimulation detection is disclosed. The method includes delivering a plurality of pacing pulses between an associated pair of electrodes of a plurality of electrodes, and, for each of the plurality of pacing pulses, measuring a corresponding diaphragm movement during delivery of the pacing pulse. The method further includes determining, for each of the plurality of pacing pulses, based on the corresponding measured diaphragm movement, whether the pacing pulse results in phrenic nerve capture, and recording, for each pacing pulse that results in phrenic nerve capture, the pair of electrodes associated with that pacing pulse.

In another aspect, a system for phrenic stimulation detection is disclosed. The system includes a catheter comprising a proximal end, a distal end, and a plurality of electrodes disposed on the distal end, at least one energy generator connected to the plurality of electrodes, a memory device, and at least one processor. The at least one processor is programmed to cause the at least one energy generator to deliver a plurality of pacing pulses, each pacing pulse delivered between an associated pair of electrodes of the plurality of electrodes. For each of the plurality of pacing pulses, the at least one processor is programmed to measure a corresponding diaphragm movement during delivery of the pacing pulse. The at least one processor is further programmed to determine, for each of the plurality of pacing pulses, based on the corresponding measured diaphragm movement, whether the pacing pulse results in phrenic nerve capture. For each pacing pulse that results in phrenic nerve capture, the pair of electrodes associated with that pacing pulse is recorded in the memory.

The foregoing and other aspects, features, details, utilities, and advantages of the present disclosure will be apparent from reading the following description and claims, and from reviewing the accompanying drawings.

### BRIEF DESCRIPTION OF THE DRAWINGS

FIG. 1 is a schematic and block diagram view of a system for electroporation therapy and automatic phrenic nerve monitoring, according to an embodiment.

FIGS. 2A and 2B are views of one embodiment of a catheter assembly that may be used with the system shown in FIG. 1.

FIGS. 3A-3C are views of alternative embodiments of a catheter assembly that may be used with the system shown in FIG. 1.

FIG. 4 is a view of an alternative embodiment of a catheter assembly that may be used with the system shown in FIG. 1.

FIG. 5 is a flow diagram of one embodiment of a method for phrenic nerve monitoring that may be implemented using the system shown in FIG. 1.

FIG. 6 is a graph illustrating sensor movement with and without electrode stimulation, according to an embodiment.

FIGS. 7A-7C are diagrams of an electrode assembly undergoing an automated stimulation protocol, according to one embodiment.

FIGS. 8A and 8B are diagrams of an electrode assembly delivering therapy, according to one embodiment.

### DETAILED DESCRIPTION

The present disclosure provides systems and methods for automatic detection of phrenic nerve stimulation. A plurality of pacing pulses are delivered between an associated pair of electrodes of a plurality of electrodes. Next, for each of the plurality of pacing pulses, a corresponding diaphragm movement during delivery of the pacing pulse is measured. For each of the plurality of pacing pulses, it is determined, based on the corresponding measured diaphragm movement, whether the pacing pulse results in phrenic nerve capture. For each pacing pulse that results in phrenic nerve capture, the pair of electrodes associated with that pacing pulse is recorded.

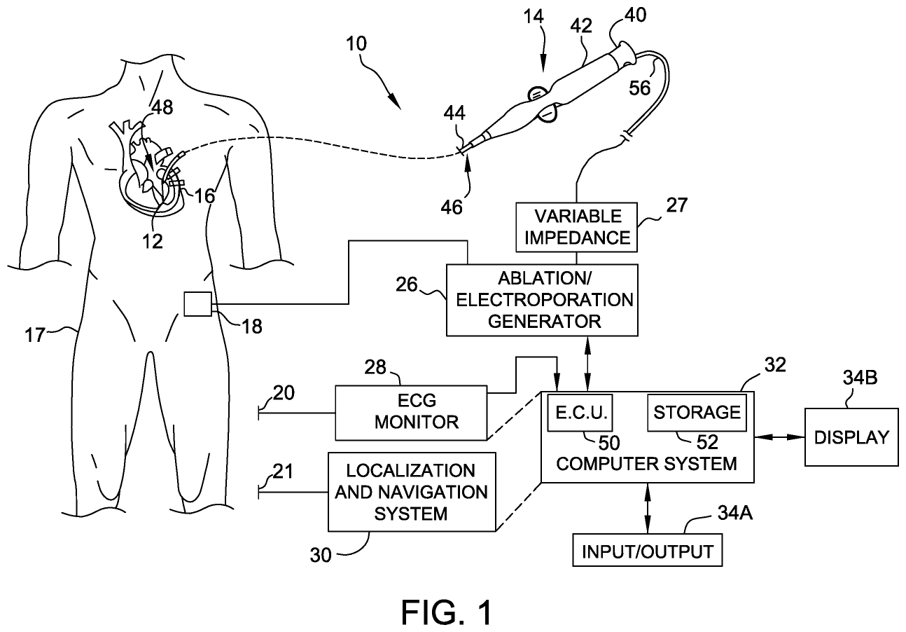

FIG. 1 is a schematic and block diagram view of a system 10 for electroporation therapy. In general, system 10 includes a catheter electrode assembly 12 disposed at a distal end 48 of a catheter 14. As used herein, “proximal” refers to a direction toward the end of the catheter near the clinician and “distal” refers to a direction away from the clinician and (generally) inside the body of a patient. The electrode assembly includes one or more individual, electrically isolated electrode elements. Each electrode element, also referred to herein as a catheter electrode, is individually wired such that it can be selectively paired or combined with any other electrode element to act as a bipolar or a multi-polar electrode.

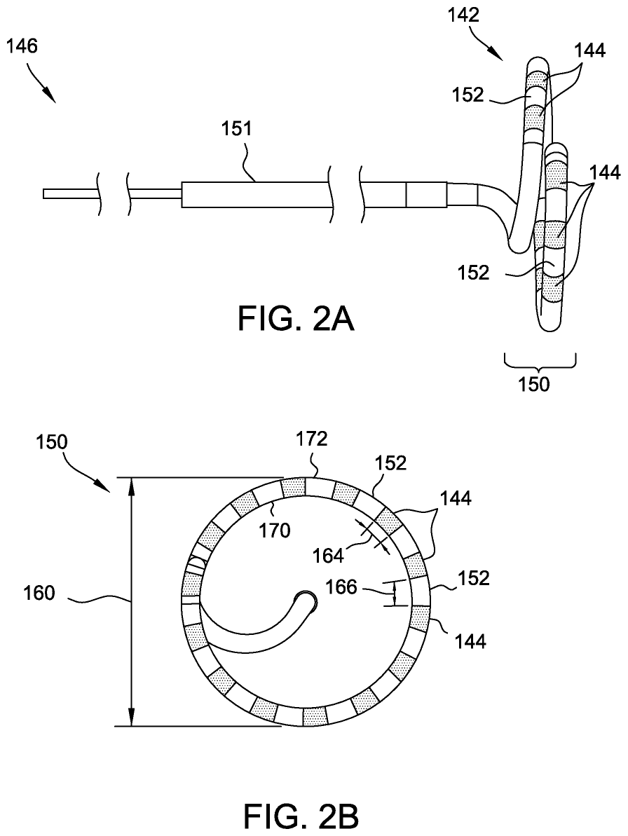

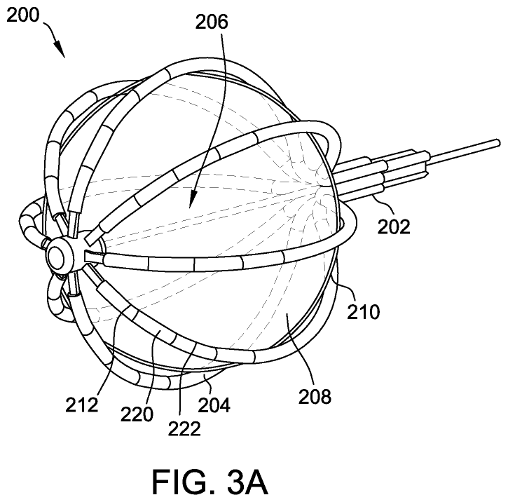

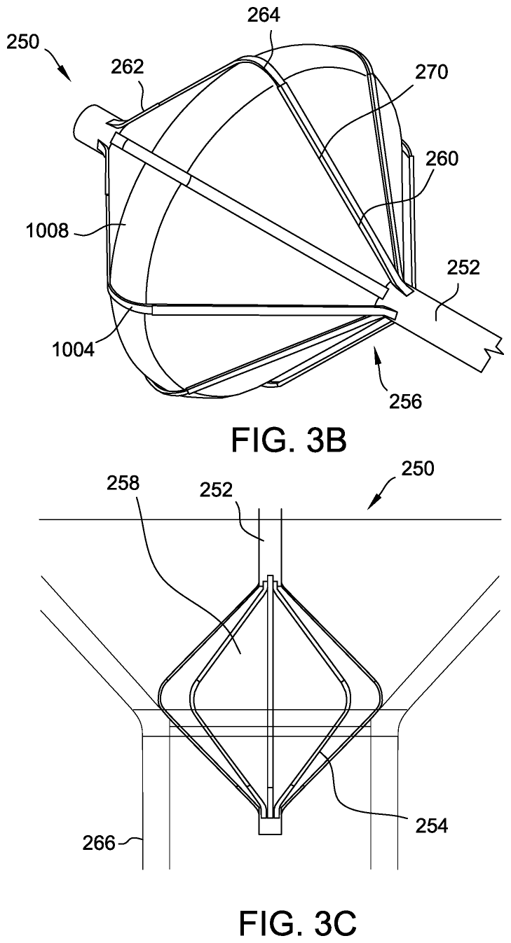

System 10 may be used for irreversible electroporation (IRE) to destroy tissue. In particular, system 10 may be used for electroporation-induced therapy that includes delivering electrical current in such a manner as to directly cause an irreversible loss of plasma membrane (cell wall) integrity leading to its breakdown and cell destruction. This mechanism of cell destruction may be viewed as an “outside-in” process, meaning that the disruption of the outside plasma membrane of the cell causes detrimental effects to the inside of the cell. Typically, for classical plasma membrane electroporation, electric current is delivered as a pulsed electric field in the form of short-duration pulses (e.g., having a 10 nanosecond (ns) to 100 millisecond (ms) duration) between closely spaced electrodes capable of delivering an electric field strength of about 0.1 to 3.0 kilovolts/centimeter (kV/cm). In some alternative embodiments, the electric field strength may be higher (e.g., greater than or equal to 3.0 kV/cm). System 10 may be used for high output (e.g., high voltage and/or high current) electroporation procedures. Further, system 10 may be used with a loop catheter such as that depicted in FIGS. 2A and 2B, and/or with a basket catheter such as those depicted in FIGS. 3A-3C. In some embodiments, system 10 is used for reversible electroporation instead of or in addition to irreversible electroporation.

In one embodiment, stimulation is delivered selectively (e.g., between pairs of electrodes) on catheter 14. Further, the electrodes on catheter 14 may be switchable between being connected to a 3D mapping system and being connected to an electroporation generator.

Irreversible electroporation through a multi-electrode catheter may enable pulmonary vein isolation in as few as one shock per vein, which may produce much shorter procedure times compared to sequentially positioning a radiofrequency (RF) ablation tip around a vein.

It should be understood that while the energization strategies are described as involving DC pulses, embodiments may use variations and remain within the spirit and scope of the disclosure. For example, exponentially-decaying pulses, exponentially-increasing pulses, and combinations may be used. Further, in some embodiments, AC pulses may also be used.

Further, it should be understood that the mechanism of cell destruction in electroporation is not primarily due to heating effects, but rather to cell membrane disruption through application of a high-voltage electric field. Thus, electroporation may avoid some possible thermal effects that may occur when using radio frequency (RF) energy. This “cold therapy” thus has desirable characteristics.

With this background, and now referring again to FIG. 1, system 10 includes a catheter electrode assembly 12 including at least one catheter electrode. Electrode assembly 12 is incorporated as part of a medical device such as a catheter 14 for electroporation therapy of tissue 16 in a body 17 of a patient. In the illustrative embodiment, tissue 16 includes heart or cardiac tissue. It should be understood, however, that embodiments may be used to conduct electroporation therapy with respect to a variety of other body tissues (e.g., renal tissue, tumors, etc.).

FIG. 1 further shows a plurality of return electrodes designated 18, 20, and 21, which are diagrammatic of the body connections that may be used by the various sub-systems included in overall system 10, such as an electroporation generator 26, an electrophysiology (EP) monitor such as an ECG monitor 28, and a localization and navigation system 30 for visualization, mapping, and navigation of internal body structures. In the example embodiment, to perform electroporation, electric fields are applied between pairs of electrodes on electrode assembly 12 (in a bipolar approach), as described further below. Alternatively, electric fields may be applied between an external return electrode (such as return electrode 18) and one or more electrodes on electrode assembly 12 (in a monopolar approach).

In the illustrated embodiment, return electrodes 18, 20, and 21 are patch electrodes. It should be understood that the illustration of a single patch electrode is diagrammatic only (for clarity) and that such sub-systems to which these patch electrodes are connected may, and typically will, include more than one patch (body surface) electrode, and may include split patch electrodes (as described herein). In other embodiments, return electrodes 18, 20, and 21 may be any other type of electrode suitable for use as a return electrode including, for example, one or more catheter electrodes. Return electrodes that are catheter electrodes may be part of electrode assembly 12 or part of a separate catheter or device (not shown). System 10 may further include a main computer system 32 (including an electronic control unit 50 and data storage-memory 52), which may be integrated with localization and navigation system 30 in certain embodiments. System 32 may further include conventional interface components, such as various user input/output mechanisms 34A and a display 34B, among other components.

Electroporation generator 26 is configured to energize the electrode element(s) in accordance with an electroporation energization strategy, which may be predetermined or may be user-selectable. For electroporation therapy, generator 26 may be configured to produce an electric current that is delivered via electrode assembly 12 as a pulsed electric field in the form of short-duration DC pulses (e.g., a nanoseconds to several milliseconds duration, or any duration suitable for electroporation) between closely spaced electrodes capable of delivering an electric field strength (i.e., at the tissue site) of about 0.1 to 3.0 kV/cm. In some alternative embodiments, the electric field strength may be higher (e.g., greater than or equal to 2.0 kV/cm). The amplitude and pulse width needed for irreversible electroporation are inversely related. That is, as pulse widths are decreased, the amplitude may generally be increased to achieve chronaxie.

Electroporation generator 26, sometimes also referred to herein as a DC energy source, is a biphasic electroporation generator 26 configured to generate a series of DC energy pulses that all produce current in two directions (i.e., positive and negative pulses). In other embodiments, electroporation generator is a monophasic or polyphasic electroporation generator. In some embodiments, electroporation generator 26 is configured to output energy in DC pulses at selectable energy levels, such as fifty joules, one hundred joules, two hundred joules, and the like. Other embodiments may have more or fewer energy settings and the values of the available setting may be the same or different. For successful electroporation, some embodiments utilize the two hundred joule output level. For example, electroporation generator 26 may output a DC pulse having a peak magnitude from about 300 Volts (V) to about 3,200 V. Other embodiments may output any other suitable positive or negative voltage.

In some embodiments, a variable impedance 27 allows the impedance of system 10 to be varied to limit arcing. Moreover, variable impedance 27 may be used to change one or more characteristics, such as amplitude, duration, pulse shape, and the like, of an output of electroporation generator 26. Although illustrated as a separate component, variable impedance 27 may be incorporated in catheter 14 or generator 26.

With continued reference to FIG. 1, as noted above, catheter 14 may include functionality for electroporation and in certain embodiments also additional ablation functions (e.g., RF ablation). It should be understood, however, that in those embodiments, variations are possible as to the type of ablation energy provided (e.g., cryoablation, ultrasound, etc.).

In the illustrative embodiment, catheter 14 includes a cable connector or interface 40, a handle 42, and a shaft 44 having a proximal end 46 and a distal 48 end. Catheter 14 may also include other conventional components not illustrated herein such as a temperature sensor, additional electrodes, and corresponding conductors or leads. Connector 40 provides mechanical and electrical connection(s) for cable 56 extending from generator 26. Connector 40 may include conventional components known in the art and as shown is disposed at the proximal end of catheter 14.

Handle 42 provides a location for the clinician to hold catheter 14 and may further provide means for steering or the guiding shaft 44 within body 17. For example, handle 42 may include means to change the length of a guidewire extending through catheter 14 to distal end 48 of shaft 44 or means to steer shaft 44. Moreover, in some embodiments, handle 42 may be configured to vary the shape, size, and/or orientation of a portion of the catheter, and it will be understood that the construction of handle 42 may vary. In an alternate embodiment, catheter 14 may be robotically driven or controlled. Accordingly, rather than a clinician manipulating a handle to advance/retract and/or steer or guide catheter 14 (and shaft 44 thereof in particular), a robot is used to manipulate catheter 14. Shaft 44 is an elongated, tubular, flexible member configured for movement within body 17. Shaft 44 is configured to support electrode assembly 12 as well as contain associated conductors, and possibly additional electronics used for signal processing or conditioning. Shaft 44 may also permit transport, delivery and/or removal of fluids (including irrigation fluids and bodily fluids), medicines, biologics, and/or surgical tools or instruments. Shaft 44 may be made from conventional materials such as polyurethane and defines one or more lumens configured to house and/or transport electrical conductors, fluids or surgical tools, as described herein. Shaft 44 may be introduced into a blood vessel or other structure within body 17 through a conventional introducer. Shaft 44 may then be advanced/retracted and/or steered or guided through body 17 to a desired location such as the site of tissue 16, including through the use of guidewires or other means known in the art.

Localization and navigation system 30 may be provided for visualization, mapping and navigation of internal body structures. Localization and navigation system 30 may include conventional apparatus known generally in the art. For example, localization and navigation system 30 may be substantially similar to the EnSite Precision™ System, commercially available from Abbott Laboratories, and as generally shown in commonly assigned U.S. Pat. No. 7,263,397 titled “Method and Apparatus for Catheter Navigation and Location and Mapping in the Heart”, the entire disclosure of which is incorporated herein by reference. In another example, localization and navigation system 30 may be substantially similar to the EnSite X™ Mapping System, as generally shown in U.S. Pat. App. Pub. No. 2020/0138334 titled “Method for Medical Device Localization Based on Magnetic and Impedance Sensors”, the entire disclosure of which is incorporated herein by reference. It should be understood, however, that localization and navigation system 30 is an example only, and is not limiting in nature. Other technologies for locating/navigating a catheter in space (and for visualization) are known, including for example, the CARTO navigation and location system of Biosense Webster, Inc., the Rhythmia® system of Boston Scientific Scimed, Inc., the KODEX® system of Koninklijke Philips N.V., the AURORA® system of Northern Digital Inc., or commonly available fluoroscopy systems.

In this regard, some of the localization, navigation and/or visualization systems may include one or more sensors for producing signals indicative of catheter location information, and may include, for example, one or more electrodes in the case of an impedance-based localization system, or alternatively, one or more coils (i.e., wire windings) configured to detect one or more characteristics of a magnetic field, for example in the case of a magnetic-field based localization system. As yet another example, system 10 may utilize a combination electric field-based and magnetic field-based system as generally shown with reference to U.S. Pat. No. 7,536,218 entitled “Hybrid Magnetic-Based and Impedance Based Position Sensing,” the disclosure of which is incorporated herein by reference in its entirety.

Pulsed field ablation (PFA), which is a methodology for achieving irreversible electroporation, may be implemented using the systems and methods described herein. In some cases, PFA may be used at specific cardiac tissue sites such as the pulmonary veins to perform a pulmonary vein isolation (PVI). In the example embodiment, electric fields are applied between adjacent electrodes (in a bipolar approach). Alternatively, electric fields may be applied between one or more electrodes and a return patch (in a monopolar approach). There are advantages and disadvantages to each of these approaches.

For lesion size and proximity, the monopolar approach has a wider range of effect, and can potentially create deeper lesions with the same applied voltage. Further, the monopolar approach may be able to create lesions from a distance (e.g., generally proximate, but not necessarily contacting tissue). The bipolar approach may create smaller lesions, requiring closer proximity or contact with tissue to create transmural lesions. However, the monopolar approach may create larger lesions than are necessary, while the lesions generated using the bipolar approach may be more localized.

Due to a wider range of effect, the monopolar approach may cause unwanted skeletal muscle and/or nerve activation. In contrast, the bipolar approach has a constrained range of effect proportional to electrode spacing on the lead, and is less likely to depolarize cardiac myocytes or nerve fibers.

To monitor operation of system 10, one or more impedances between catheter electrodes 144 and/or return electrodes 18, 20, and 21 may be measured. For example, for system 10, impedances may be measured as described in U.S. Patent Application Publication No. 2019/0117113, filed on Oct. 23, 2018, U.S. Patent Application Publication No. 2019/0183378, filed on Dec. 19, 2018, and International Patent Application Publication No. WO 2021/236341, filed on May 6, 2021, all of which are incorporated by reference herein in their entirety.

FIGS. 2A and 2B are views of one embodiment of a catheter assembly 146 that may be used with catheter 14 in system 10. Catheter assembly 146 may be referred to as a loop catheter. Those of skill in the art will appreciate that, in other embodiments, any suitable catheter may be used. Specifically, FIG. 2A is a side view of catheter assembly 146 with a variable diameter loop 150 at a distal end 142. FIG. 2B is an end view of variable diameter loop 150 of catheter assembly 146. Those of skill in the art will appreciate that the methods and systems described herein may be implemented using any suitable catheter (e.g., fixed loop catheters, linear catheters, basket catheter, etc.). As shown in FIGS. 2A and 2B, variable diameter loop 150 is coupled to a distal section 151 of shaft 44.

Variable diameter loop 150 is selectively transitionable between an expanded (also referred to as “open”) diameter 160 (shown in FIG. 2A) and a retracted (also referred to as “closed”) diameter 160 (not shown). In the example embodiment, an expanded diameter 160 is twenty eight mm and a retracted diameter 160 is fifteen mm. In other embodiments, diameter 160 may be variable between any suitable open and closed diameters 160.

In the embodiment shown, variable diameter loop 150 includes fourteen catheter electrodes 144 substantially evenly spaced around the circumference of variable diameter loop 150 in the expanded configuration. In the retracted configuration, one or more of electrodes 144 may overlap. In other embodiments, other arrangements of catheter electrodes 144 may be implemented. For example, in one embodiment, variable diameter loop 150 includes twelve catheter electrodes 144.

Catheter electrodes 144 are platinum ring electrodes configured to conduct and/or discharge electrical current in the range of one thousand volts and/or ten amperes. In other embodiments, variable diameter loop 150 may include any suitable number of catheter electrodes 144 made of any suitable material. Catheter electrodes 144 may include any catheter electrode suitable to conduct high voltage and/or high current (e.g., in the range of one thousand volts and/or ten amperes). Each catheter electrode 144 is separated from each other catheter electrode by an insulated gap 152. In the example embodiment, each catheter electrode 144 has a same length 164 (shown in FIG. 2B) and each insulated gap 152 has a same length 166 as each other gap 152. Length 164 and length 166 are both about 2.5 mm in the example embodiment. In other embodiments, length 164 and length 166 may be different from each other. Moreover, in some embodiments, catheter electrodes 144 may not all have the same length 164 and/or insulated gaps 152 may not all have the same length 166. In some embodiments, catheter electrodes 144 are not spaced evenly around the circumference of variable diameter loop 150.

Diameter 160 and catheter electrode 144 spacing may be developed to provide a targeted range of energy density to tissue, as well as to provide sufficient electroporation coverage for different human anatomic geometries. In general, a sufficient number of electrodes 144 with appropriate lengths 164 are desired to provide substantially even and continuous coverage around the circumference of variable diameter loop 150, while still allowing enough flexibility to allow variable diameter loop 150 to expand and contract to vary diameter 160 to the desired extremes.

As mentioned above, length 164 of catheter electrodes 144 may be varied. Increasing length 164 of catheter electrodes 144 may increase coverage of electrodes 144 around the circumference of variable diameter loop 150 while also decreasing current density (by increasing the surface area) on electrodes 144, which may help prevent arcing during electroporation operations. Increasing length 164 too much, however, may prevent variable diameter loop 150 from forming a smooth circular shape and may limit the closed diameter 160 of variable diameter loop 150. Additionally, too great a length 164 may increase the surface area of catheter electrodes 144 to a point that the current density applied to catheter electrodes 144 by a power source is below the minimum current density needed for successful therapy. Conversely, decreasing length 164 decreases the surface area, thereby increasing the current density (assuming no other system changes) on catheter electrodes 144. As discussed above, greater current densities may lead to increased risk of arcing during electroporation, and may result in larger additional system resistances needing to be added to prevent arcing. Moreover, in order to get a desired, even coverage about the circumference of variable diameter loop 150, more catheter electrodes 144 may be needed if length 164 is decreased. Increasing the number of catheter electrodes 144 on variable diameter loop 150 may prevent variable diameter loop 150 from being able to be contracted to a desired minimum diameter 160.

FIG. 3A is a perspective view of an alternative catheter assembly 200 that may be used with catheter 14. Catheter assembly 200 may be referred to as a basket catheter. Catheter assembly 200 includes a shaft 202 and a plurality of splines 204 surrounding a distal portion 206 of shaft 202. In this embodiment, catheter assembly 200 also includes a balloon 208 enclosed by splines 204. Balloon 208 may be selectively inflated to fill the space between splines 204. Notably, balloon 208 functions as an insulator, and generally reduces energy losses, which may result in increased lesion size.

Each spline 204 includes a proximal end 210 coupled to shaft 202 and a distal end 212 coupled to shaft 202. From proximal end 210 to distal end 212, spline 204 has an arcuate shape that extends radially outward.

In this embodiment, each spline 204 includes one or a plurality of individual electrodes 220. For example, each spline 204 may include an elastic material (e.g., Nitinol) covered in a polymer tube 222, with individual electrodes 220 attached to an exterior of polymer tube 222. In the embodiment shown, each spline 204 includes two electrodes 220. Further, as shown in FIG. 2, electrodes 220 are generally positioned closer to distal end 212 than proximal end 210 to correspond to portions of spline 204 that will contact the pulmonary vein.

Alternatively, each spline 204 may include any suitable number and arrangement of electrodes 220. For example, in some embodiments, each spline 204 includes four electrodes 220.

In this embodiment, alternating splines 204 alternate polarities. That is, electrodes 220 on a particular spline 204 have the same polarity, but electrodes 220 on a particular spline 204 have a different polarity than electrodes 220 on adjacent splines 204. Alternatively, any suitable polarization scheme may be used. During delivery, splines 204 may be collapsed in towards shaft 202. Subsequently, to perform ablation, splines 204 are deployed to extend radially outward.

Splines 204 may all have the same length, or at least some of splines 204 may have different lengths. Further, insulating material on each spline 204 may have the same length, or at least some splines 204 may have insulating material with different lengths. In addition, in some embodiments, catheter assembly 200 includes a distal electrode (not shown) positioned distal of splines 204. The distal electrode may be used to perform point ablation (e.g., by creating a bipole between the distal electrode and one of splines 204), and/or may be used for visualization/mapping purposes (e.g., using the distal electrode in combination with an electrode on shaft 202).

FIG. 3B is a perspective view of an alternative catheter assembly 250 that may be used with catheter 14, and FIG. 3C is a side schematic view of catheter assembly 250. Like catheter assembly 200 (shown in FIG. 3A), catheter assembly 250 may be referred to as a basket assembly.

Catheter assembly 250 includes a shaft 252 and a plurality of splines 254 surrounding a distal portion 256 of shaft 252. In this embodiment, catheter assembly 250 includes a balloon 258 enclosed by splines 254. Balloon 258 may be selectively inflated to occupy the space between splines 254. Notably, balloon 258 functions as an insulator, and generally reduces energy, which may result in increased lesion size.

Each spline 254 includes a proximal end 260 coupled to shaft 252 and a distal end 262 coupled to shaft 252. From proximal end 260, spline 1004 extends radially outward to an inflection point 264, and then extends radially inward to distal end 262. FIG. 3C shows catheter assembly 250 positioned within the pulmonary vein 266.

A body of each spline 254 is made of an elastic material (e.g., Nitinol), and functions as a relatively large electrode. In this embodiment, alternating splines 254 alternate polarities. That is, each positive spline 254 is positioned between two negative splines 254 and vice-versa. Alternatively, any suitable polarization scheme may be used.

To control the ablation zone of each spline 254, portions of each spline 254 may be covered with insulating material 270 (e.g., heat-shrink or polymer tubing or spray or dip coat with polyimide or PEBAX), and the exposed portions of splines 254 function as electrodes. In the embodiment shown in FIGS. 3B and 3C, inflection point 264 and portions of spline 254 between inflection point 264 and distal end 262 are generally exposed, while portions of spline 254 between inflection point 264 and proximal end 260 are generally insulated. This results in the portions of spline 254 that contact pulmonary vein 266 being exposed (see FIG. 3C). Alternatively, any suitable insulation configuration may be used.

During delivery, splines 254 and balloon 258 may be collapsed. To perform ablation, splines 254 are deployed with inflection points 264 extending radially outward, and balloon 258 is selectively inflated to occupy the space between splines 254.

The combination of balloon 258 and splines 254 facilitates straightforward delivery and deployment of catheter assembly 250. Further, balloon 258 drives more energy into ablated tissue, and stabilizes splines 254 to prevent lateral movement. In addition, using splines 254 as electrodes instead of individual smaller electrodes may facilitate reducing the cost and increasing the reliability of catheter assembly 250.

Splines 254 may all have the same length, or at least some of splines 254 may have different lengths. Further, insulating material 270 on each spline 254 may have the same length, or at least some splines 254 may have insulating material 270 with different lengths. In addition, in some embodiments, catheter assembly 250 includes a distal electrode (not shown) positioned distal of splines 254. The distal electrode may be used to perform point ablation (e.g., by creating a bipole between the distal electrode and one of splines 254), and/or may be used for visualization/mapping purposes (e.g., using the distal electrode in combination with an electrode on shaft 252).

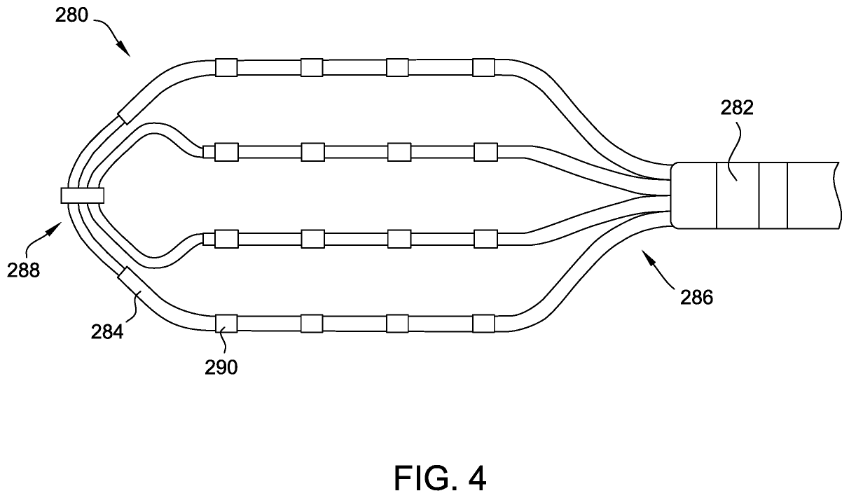

FIG. 4 is a side view of an alternative catheter assembly 280 that may be used with catheter 14. Catheter assembly 280 may be referred to as a grid assembly. As shown in FIG. 4, catheter assembly 280 is coupled to a distal section 282 of a shaft, such as shaft 44 (shown in FIG. 1).

Catheter assembly 280 includes a plurality of splines 284 extending from a proximal end 286 to a distal end 288. Each spline 284 includes a plurality of electrodes 290. In the embodiment shown in FIG. 4, catheter assembly 280 includes four splines 284, and each spline 284 includes four electrodes 290, such that electrodes 290 form a grid configuration. Accordingly, catheter assembly 280 provides a four by four grid of electrodes 290. In one embodiment, the spacing between each pair of adjacent electrodes 290 is approximately 4 millimeters (mm) such that the dimensions of the grid of electrodes 290 are approximately 12 mm×12 mm. Alternatively, catheter assembly 280 may include any suitable number of splines 284, any suitable number of electrodes 290, and/or any suitable arrangement of electrodes 290. For example, in some embodiments, the spacing between each pair of adjacent electrodes is approximately 2 millimeters (mm). Further, in some embodiments, catheter assembly 280 may include, for example, fifty-six electrodes arranged in a 7×8 grid.

Using catheter assembly 280, lesions may be generated at individual electrodes 290 using a monopolar approach (e.g., by applying a voltage between individual electrodes 290 and a return patch) or generated between pairs of electrodes 290 using a bipolar approach. Lesions may be generating within an anatomy by selectively energizing electrodes in a particular configuration and/or pattern (e.g., including energizing individual electrodes 290 independent of one another, or energizing multiple electrodes 290 simultaneously).

Those of skill the art will appreciate that catheter assembly 146 (shown in FIGS. 2A and 2B), catheter assembly 200 (shown in FIG. 3A), catheter assembly 250 (shown in FIGS. 3B and 3C), and catheter assembly 280 (shown in FIG. 4) are merely examples. Notably, the systems and methods described herein may be implemented using any suitable catheter assembly.

For electroporation therapy, waveforms are generated using a pulse generator (e.g., electroporation generator 26 (shown in FIG. 1)) and applied between pairs of catheter electrodes (i.e., a bipolar approach) or between individual catheter electrodes and a return patch (i.e., a monopolar approach). The waveforms may be monophasic, biphasic (i.e., having both a positive pulse and a negative pulse), or polyphasic. Further, the waveforms may include one or more bursts of pulses (with each burst including multiple pulses). Further, the waveforms are defined by multiple parameters (e.g., pulse width, pulse amplitude, frequency, etc.).

To generate different waveforms, the pulse generator selectively connects different electrodes to different voltage levels. In at least some known systems, a first subset of electrodes is selectively connectable to a first voltage level (e.g., a positive voltage), and a second subset of electrodes is selectively connectable to a second voltage level (e.g., a negative voltage).

The voltage threshold for cardiac cells is approximately 400 Volts/centimeter (V/cm) (e.g., field strengths below 400 V/cm may be insufficient to damage cardiac cells). In general, PFA therapy spans between electrodes and protrudes deep into the tissue, with voltage gradients surpassing 400 V/cm generally being achieved at locations near the electrodes. Given the relatively high field strength during PFA, if these electrodes are sufficiently proximate the phrenic nerve, which provides innervation for the diaphragm, there is potential for acute phrenic stunning and/or long-term phrenic damage as a result of the therapy. Accordingly, to reduce the potential for phrenic impact, a physician may avoid ablating in areas near the phrenic nerve or use a lower dosage when ablating near the phrenic nerve. Methods for automatic phrenic nerve monitoring are described herein. The automatic phrenic nerve monitoring may be implemented using, for example, system 10 (shown in FIG. 1). The automatic phrenic nerve monitoring described herein enables physicians to detect the phrenic nerve and deliver an ablation therapy which eliminates or greatly reduces damage to the phrenic nerve.

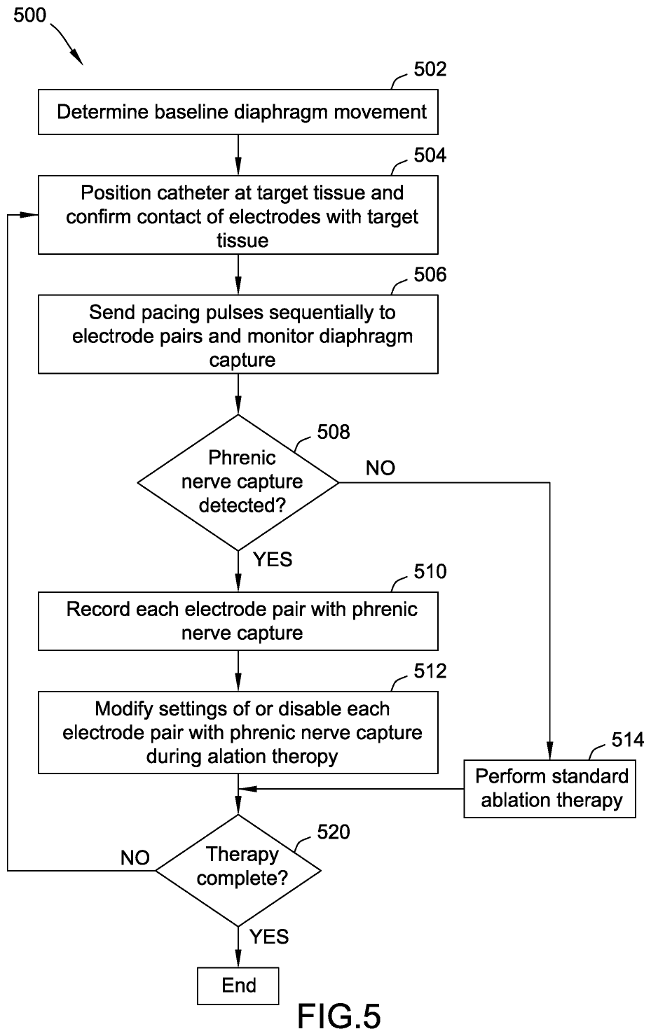

FIG. 5 is flow diagram 500 of one embodiment of a method for automatic phrenic nerve monitoring. Initially, a patient's baseline diaphragm movement may be monitored at block 502. In some embodiments, diaphragm movement may be measured using one or more sensors that are placed on the patient's body. To determine the baseline diaphragm movement, the one or more sensors may continuously monitor the diaphragm movement for a predetermined period of time. The one or more sensors may include, but are not limited to impedance sensors, magnetic sensors (e.g., an anterior patient reference sensor (PRS-A)), fiber optic sensors, accelerometers, and/or or any other sensor suitable for sensing diaphragm movement. In some embodiments, the impedance and/or magnetic sensors of localization system 30 of system 10 may be used to capture a baseline diaphragm movement. In other embodiments, the sensors used to capture the baseline diaphragm movement are distinct from localization system 30. In some embodiments, the one or more sensors may include a body patch, including but not limited to an ECG patch, an EnSite™ NavX™ patch available from Abbott Laboratories, and/or a new patch. In some embodiments, the one or more sensors include two body patches. In further embodiments, the two body patches are placed on or near the chest and/or back of the patient.

Diaphragm movement may be measured using any suitable systems or methods. For example, in some embodiments, diaphragm movement is measured using one or more imaging systems. For example, in some embodiments, diaphragm movement is measured using a fluoroscopy system, a transesophageal echocardiogram (TEE) system, and/or an intracardiac echocardiography (ICE) system. Additionally, or alternatively, diaphragm movement is measured using a video or still images from a video, followed by imaging processing (e.g., processing the video and/or images through an algorithm).

In some embodiments, diaphragm movement is measured using a navigation system. In further embodiments, diaphragm movement is measured using a three-dimensional navigation system. For example, in some embodiments, diaphragm movement is measured using a magnetic-based, impedance-based, fiber optic-based, and/or radiofrequency-based navigation system.

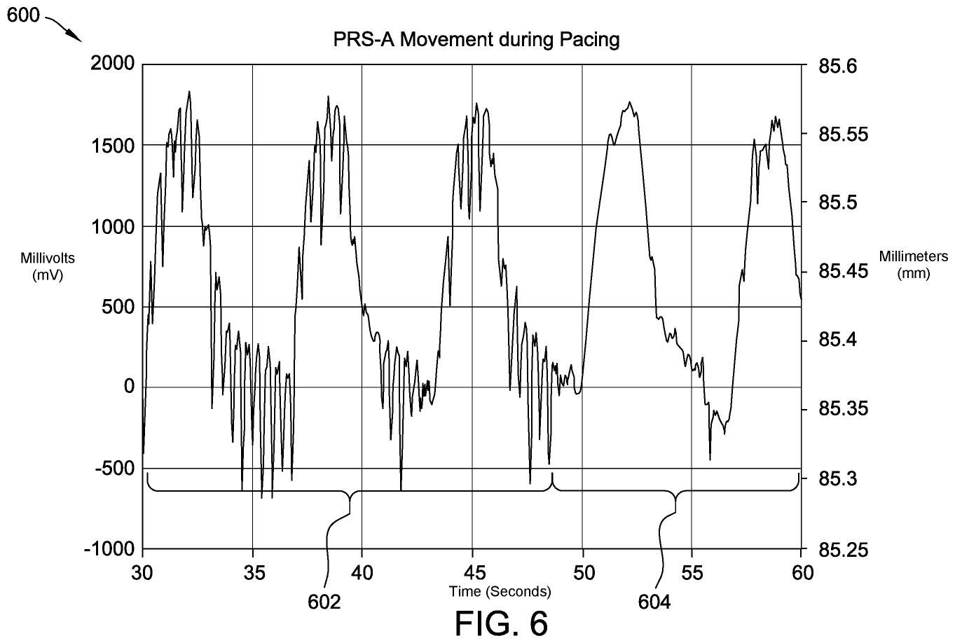

FIG. 6 is graph 600 of movement detected using a PRS-A sensor with and without stimulation. More particularly, first section 602 of the graph illustrates movement in millimeters (mm) over time in seconds detected using a PRS-A sensor while pacing pulses are being delivered to the electrodes of the electrode assembly. Second section 604 of the graph illustrates movement detected using the PRS-A sensor while no pacing pulses are being delivered to the electrodes of the electrode assembly. Accordingly, second section 604 may be used to establish a baseline diaphragm movement of a patient.

Returning to FIG. 5, catheter 14 may be positioned at or proximate to a target tissue at block 504. For example, in some embodiments, catheter 14 may be placed at or near the pulmonary vein for pulmonary vein isolation. The distal end of catheter 14 may include electrode assembly 12 including a plurality of electrodes, as described above. In some embodiments, after the catheter is positioned at the target tissue, the contact of the electrodes with the target tissue may be assessed. In some embodiments, the contact is assessed via visualization methods. For example, a 3D map of the target tissue with the catheter superimposed thereon may be generated by the localization and navigation system 30 and displayed on the user interface of display 34B. The operator may then use the 3D map to visually assess whether the catheter is in contact with the target tissue. In other embodiments, the contact may be confirmed using impedance sensing, optic-based sensing, contact force sensing, or any other contact sensing mechanisms known in the art.

At block 506, pacing pulses are delivered to sequentially to the electrodes of electrode assembly 12. In a bipolar approach, the pacing pulses may be applied between pairs of electrodes on electrode assembly 12. The pairs of electrodes may include electrodes that are adjacent to each other. In a monopolar approach, the pacing pulses are delivered to a single electrode of electrode assembly 12. The pacing pulses may be delivered sequentially to the electrodes of electrode assembly 12 for a predetermined period of time. For example, in some embodiments, the pacing pulses are delivered for a period of 500 milliseconds (ms) or less.

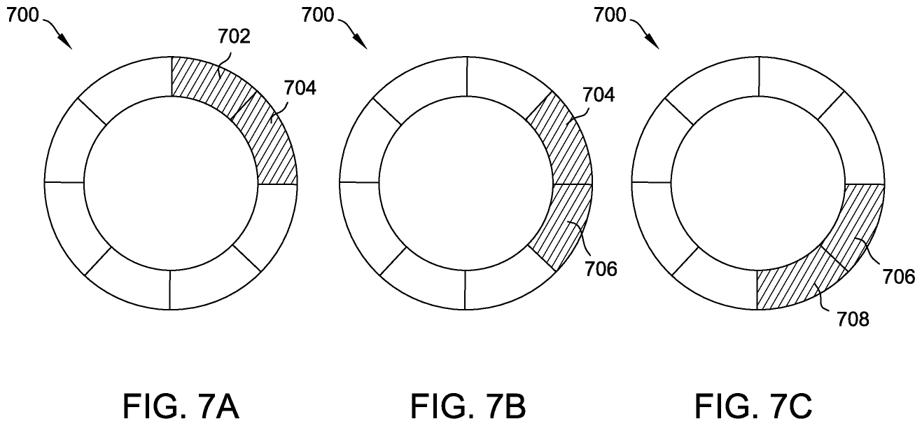

FIGS. 7A-7C are diagrams of pacing pulses being sequentially delivered to an electrode assembly 700 at a distal end of a catheter, according to one embodiment. In the embodiment illustrated in FIGS. 7A-7C, the distal end of the catheter includes eight electrodes disposed in a loop pattern intended for pulmonary vein isolation, similar to the catheter depicted in FIGS. 2A-2B. However, those of skill in the art will appreciate that the electrodes may be arranged in any suitable configuration, such as those depicted in FIGS. 3A-3C and FIG. 4. Returning to FIGS. 7A-7C, bipolar pacing pulses are generated and delivered to each pair of adjacent electrodes in a sequential order. For example, in FIG. 7A, pacing pulses are delivered between a first electrode 702 and a second electrode 704. Next, pacing pulses are delivered between the second electrode 704 and a third electrode 706, as shown in FIG. 7B. Pacing pulses may then be delivered between the third electrode 706 and a fourth electrode 708, as shown in FIG. 7C, and so forth, until pacing pulses have been delivered between every pair of adjacent of electrodes in electrode assembly 700. In some embodiments, the pacing pulses may be delivered to each electrode pair of electrode assembly 700 for a predetermined period of time (e.g., 500 ms), as discussed above.

Returning to FIG. 5, in some embodiments, ablation/electroporation generator 26 may be configured to generate and deliver the pacing pulses to the electrodes. Therefore, a single energy generator (e.g., ablation/electroporation generator 26) may function as both a pulse generator configured to deliver pacing pulses to the electrodes and an ablation/electroporation generator configured to deliver ablative energy to the electrodes. In other embodiments, system 10 includes at least two separate energy generators: an ablation/electroporation generator configured to deliver ablative energy and a separate pulse generator (not shown) that is individually connected to each of the electrodes of electrode assembly 12 and configured to generate and deliver the pacing pulses to the electrodes.

The electrical current of the pacing pulses may be sufficiently high to stimulate the phrenic nerve, but low enough as to not cause any damage to the phrenic nerve. For example, in some embodiments, the pacing pulses are 20 milliamps (mA) or less. In further embodiments, the pacing pulses are in the range of 5-10 mA. Monitoring delivery of the pacing pulses provides information as to the distance between the one or more electrodes being stimulated and the phrenic nerve. In some embodiments, one or more capture thresholds may be determined for electrodes demonstrating phrenic nerve capture. A capture threshold, as used herein, refers to the minimum amount of electrical current necessary to stimulate the phrenic nerve. A capture threshold may be determined before, during, and/or after a therapy application, as discussed in more detail below. Further, a change in the capture threshold may be indicative of an impact of ablation therapy on the phrenic nerve, which may further inform if stunning or damage could occur with future therapy applications.

At block 506, diaphragm movement is measured, the measurements coinciding with the delivery of the pacing pulses with some deterministic delay. Diaphragm movement is measured by any of the systems and methods described above (e.g., one or more sensors, one or more body patches, an imaging system, and/or a navigation system). The diaphragm movement may be continually monitored as the pacing pulses are delivered sequentially to each pair of electrodes of the electrode assembly. For example, as discussed above, in FIG. 6, first section 602 illustrates PRS-A sensor movement while pacing pulses are delivered to electrodes of the electrode assembly.

Returning to FIG. 5, at block 508, it is determined whether phrenic nerve capture was detected. Phrenic nerve capture may be detected based on the diaphragm movement while the pacing pulses are delivered at block 506. More particularly, the diaphragm movement during pacing may be compared to the baseline diaphragm movement measured at block 502 to determine whether there is phrenic nerve capture. For example, in FIG. 6, measurements taken while pacing pulses are delivered to electrodes of the electrode assembly (i.e., at first section 602) may be compared to a baseline measurement (i.e., second section 604). For example, in some embodiments, a slope and/or a frequency of the displacement of the diaphragm in comparison with the baseline diaphragm movement may be measured. A high-pass filter or a derivative of the waveform may be used to determine whether there was phrenic nerve stimulation. For example, in embodiments where one or more impedance sensors are used, the system may determine phrenic nerve stimulation based on whether a derivative of the impedance waveform shows a change of sign and whether the new sign of the derivative lasts for a predetermined period of time. In embodiments where magnetic sensors are used, the system may determine phrenic nerve stimulation based on a displacement measured by the magnetic sensors.

If phrenic nerve capture is detected, the method proceeds to block 510. At block 510, electrode pairs which were determined to have captured the phrenic nerve are logged or recorded. The pacing pulses output by those pairs of electrodes may also be logged or recorded. In some embodiments, this information may be stored in a storage device of the system, such as data storage-memory 52. Additionally, or alternatively, this information may be sent to the localization and navigation system 30 of system 10. Localization and navigation system 30 may use this information to determine phrenic nerve capture locations. Localization and navigation system 30 may further visually identify phrenic nerve capture locations on a user interface on the display 34B. For example, phrenic nerve capture locations may be displayed on a 3D map generated by localization and navigation system 30 and displayed on the user interface of display 34B. The phrenic nerve capture location may be visually identified in relation to catheter 14 and/or the target tissue.

Next, a phrenic nerve safe ablation therapy may be performed at block 512. The ablation therapy may be an electroporation therapy, such as PFA. The phrenic nerve safe ablation therapy may include modifying the pulse parameters or disabling electrodes determined to have caused phrenic nerve capture. For example, if a high voltage waveform is to be used, electrodes determined to have caused phrenic capture may be disabled from applying that waveform. If a low voltage waveform is used, then a modified waveform may be used on electrodes determined to have caused phrenic capture. For example, the modified waveform may include a lower energy waveform that will eliminate or greatly reduce any impact on the phrenic nerve.

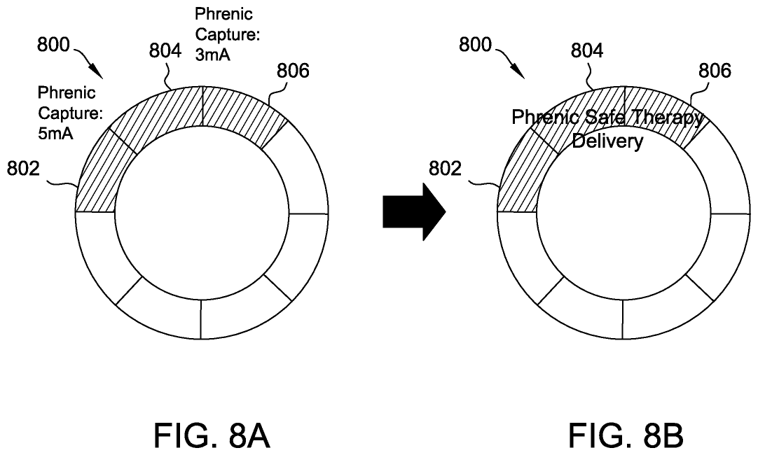

For example, in the embodiment illustrated in FIG. 8A, energy delivery between a first electrode 802 and a second electrode 804 demonstrated phrenic capture at a pacing output of 5 mA, and energy delivery between second electrode 804 and a third electrode 805 demonstrated phrenic capture at a pacing output of 3 mA. This information may be determined, for example, as discussed above in reference to blocks 502-510 of FIG. 5. This information may be recorded and optionally stored in a memory of the system. Further, during an ablation therapy, phrenic safe ablation levels are delivered to electrodes 802, 804, and 806 in FIG. 8B. The phrenic safe ablation levels may include a modified waveform, such as a lower energy waveform, as discussed above.

Returning to FIG. 5, in some embodiments, the system may automatically make the determination to disable and/or modify certain electrodes at block 512. In other embodiments, the electrodes identified as capturing the phrenic nerve and the respective pacing output may be indicated on a user interface on the display 34B. For example, in the embodiment illustrated in FIGS. 8A and 8B, the user interface may indicate that the pair of first electrode 802 and second electrode 804, and the pair of second electrode 804 and third electrode 806 demonstrated phrenic nerve capture. The user interface may further indicate that the pair of first electrode 802 and second electrode 804 demonstrated phrenic capture at a pacing output of 5 mA and the pair of second electrode 804 and third electrode 806 demonstrated phrenic capture at a pacing output of 3 mA. Additionally, or alternatively, the phrenic nerve locations may be indicated on a 3D map, as discussed above. The operator may use this information to select phrenic safe ablation levels to and/or disable specific electrodes.

Returning to FIG. 5, if it is determined that there are no electrode pairs that demonstrate phrenic nerve capture at block 508, an ablation therapy may be performed at standard therapy levels at block 514.

At block 520, it is determined whether the therapy is complete. If the therapy is not complete, method 500 returns to block 504, and catheter 14 remains positioned at the target tissue site or is positioned at another target tissue site. Optionally, one or more additionally ablation therapy applications may be performed before returning to block 504. Therefore, in some embodiments, pacing occurs in between ablation therapy applications. In other embodiments, pacing occurs after a plurality of ablation therapy applications. Once therapy is complete, method 500 ends at block 522.

In some embodiments, one or more capture thresholds may be determined for electrodes demonstrating phrenic nerve capture. A capture threshold may refer to the minimum amount of electrical current necessary to stimulate the phrenic nerve. In some embodiments, a first capture threshold may be determined for an electrode pair demonstrating phrenic nerve capture before an ablation therapy application. A second capture threshold may then be determined for that electrode pair after an ablation therapy application, or in-between bursts during an ablation therapy application. The second capture threshold may be compared to the first capture threshold. If the second capture threshold is greater than the first capture threshold, this may indicate an impact of PFA therapy on the phrenic nerve. For example, this may indicate to a clinician that stunning or damage may occur with further therapy applications. In some embodiments, if there is an increase in capture threshold (i.e., the second capture threshold is greater than the first capture threshold), then the system may automatically stop delivering therapy and/or begin delivering phrenic safe therapy. Additionally, or alternatively, the increase in capture threshold may be indicated on display 34B, and the clinician may determine whether to stop the therapy and/or begin delivering phrenic safe therapy. In some embodiments, an increase in capture threshold may be also accompanied by a visual or audio alarm.

Although certain embodiments of this disclosure have been described above with a certain degree of particularity, those skilled in the art could make numerous alterations to the disclosed embodiments without departing from the spirit or scope of this disclosure. All directional references (e.g., upper, lower, upward, downward, left, right, leftward, rightward, top, bottom, above, below, vertical, horizontal, clockwise, and counterclockwise) are only used for identification purposes to aid the reader's understanding of the present disclosure, and do not create limitations, particularly as to the position, orientation, or use of the disclosure. Joinder references (e.g., attached, coupled, connected, and the like) are to be construed broadly and may include intermediate members between a connection of elements and relative movement between elements. As such, joinder references do not necessarily infer that two elements are directly connected and in fixed relation to each other. It is intended that all matter contained in the above description or shown in the accompanying drawings shall be interpreted as illustrative only and not limiting. Changes in detail or structure may be made without departing from the spirit of the disclosure as defined in the appended claims.

When introducing elements of the present disclosure or the preferred embodiment(s) thereof, the articles “a”, “an”, “the”, and “said” are intended to mean that there are one or more of the elements. The terms “comprising”, “including”, and “having” are intended to be inclusive and mean that there may be additional elements other than the listed elements.

As various changes could be made in the above constructions without departing from the scope of the disclosure, it is intended that all matter contained in the above description or shown in the accompanying drawings shall be interpreted as illustrative and not in a limiting sense.

What is claimed is:

1. A method for phrenic nerve stimulation detection, the method comprising:
delivering a plurality of pacing pulses, each pacing pulse delivered between an associated pair of electrodes of a plurality of electrodes;
for each of the plurality of pacing pulses, measuring a corresponding diaphragm movement during delivery of the pacing pulse;
determining, for each of the plurality of pacing pulses, based on the corresponding measured diaphragm movement, whether the pacing pulse results in phrenic nerve capture; and
recording, for each pacing pulse that results in phrenic nerve capture, the pair of electrodes associated with that pacing pulse.

2. The method of claim 1, further comprising delivering pulsed field ablation therapy using at least some of the plurality of electrodes.

3. The method of claim 2, wherein delivering pulsed field ablation therapy comprises disabling a pair of the plurality of electrodes that is associated with a pacing pulse that results in phrenic nerve capture, such that the pair of the plurality of electrodes that is associated with a pacing pulse that results in phrenic nerve capture is not used to deliver the pulsed field ablation therapy.

4. The method of claim 2, wherein delivering pulsed field ablation comprises delivering a phrenic safe waveform between a pair of the plurality of electrodes that is associated with a pacing pulse that results in phrenic nerve capture.

5. The method of claim 1, further comprising indicating on a display device a pair of the plurality of electrodes that is associated with a pacing pulse that results in phrenic nerve capture.

6. The method of claim 1, wherein determining whether the pacing pulse results in phrenic capture comprises determining whether the pacing pulse results in phrenic nerve capture based on a slope or a frequency of the corresponding measured diaphragm movement.

7. The method of claim 1, wherein each pacing pulse has a current of 20 milliamps (mA) or less.

8. The method of claim 7, wherein each pacing pulse has a current between 5 mA and 10 mA.

9. The method of claim 1, further comprising determining at least one capture threshold for a pair of the plurality of electrodes that is associated with a pacing pulse that results in phrenic nerve capture, the at least one capture threshold representing a minimum amount of electrical current necessary to stimulate the phrenic nerve.

10. The method of claim 9, wherein the at least one capture threshold includes a first capture threshold measured before an ablation therapy is applied and a second capture threshold measured after the ablation therapy is applied.

11. A system for phrenic nerve stimulation detection comprising:
a catheter comprising a proximal end, a distal end, and a plurality of electrodes disposed on the distal end;
at least one energy generator connected to the plurality of electrodes; and
at least one processor and a memory device, the at least one processor programmed to:
cause the at least one energy generator to deliver a plurality of pacing pulses, each pacing pulse delivered between an associated pair of electrodes of the plurality of electrodes;
for each of the plurality of pacing pulses, measure a corresponding diaphragm movement during delivery of the pacing pulse;
determine, for each of the plurality of pacing pulses, based on the corresponding measured diaphragm movement, whether the pacing pulse results in phrenic nerve capture; and
record in the memory, for each pacing pulse that results in phrenic nerve capture, the pair of electrodes associated with that pacing pulse.

12. The system of claim 11, further comprising a display device, wherein the processor is further configured to indicate on the display device a pair of electrodes that is associated with a pacing pulse resulting in phrenic nerve capture.

13. The system of claim 11, wherein the at least one energy generator is configured to delivery pulsed field ablation therapy using at least some of the plurality of electrodes.

14. The system of claim 13, wherein the at least one processor is programmed to disable a pair of the plurality of electrodes that is associated with a pacing pulse that results in phrenic nerve capture, such that the pair of the plurality of electrodes that is associated with a pacing pulse that results in phrenic nerve capture is not used to deliver the pulsed field ablation therapy.

15. The system of claim 13, wherein the at least one processor is programmed to cause the at least one energy generator to deliver a phrenic safe waveform between a pair of the plurality of electrodes that is associated with a pacing pulse that results in phrenic nerve capture.

16. The system of claim 11, wherein the at least one processor is programmed to determine whether the pacing pulse results in phrenic capture based on a slope or a frequency of the corresponding measured diaphragm movement.

17. The system of claim 11, wherein each pacing pulse has a current of 20 milliamps (mA) or less.

18. The system of claim 11, wherein each pacing pulse has a current between 5 mA and 10 mA.

19. The system of claim 11, wherein the at least one processor is further programmed to determine at least one capture threshold for a pair of the plurality of electrodes that is associated with a pacing pulse that results in phrenic nerve capture, the at least one capture threshold representing a minimum amount of electrical current necessary to stimulate the phrenic nerve.

20. The system of claim 11, further comprising one or more sensors configured to measure the corresponding diaphragm movement during delivery of the pacing pulse.
